(19) United States
(12) Patent Application Publication
(10) Pub. No.: US 2005/0182049 A1
(43) Pub. Date: Aug. 18, 2005

(54) Combination of gamma-aminobutyric acid modulators and 5-HT1B receptor antagonists

(75) Inventor: Harry Ralph Howard JR., Bristol, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/046,974

(22) Filed: Jan. 31, 2005

Related U.S. Application Data
(60) Provisional application No. 60/540,517, filed on Jan. 29, 2004.

(51) Int. Cl. A61K 31/541

(52) U.S. Cl. 514/227.5

(57) ABSTRACT

The present invention relates to a pharmaceutical composition for treating a disorder or condition selected from the group consisting of hypertension, depression, generalized anxiety disorder, phobias, posttraumatic stress disorder, avoidant personality disorder, sexual dysfunction, eating disorders, obesity, chemical dependencies, cluster headache, migraine, pain, Alzheimer's disease, obsessive-compulsive disorder, panic disorder, memory disorders, Parkinson's diseases, endocrine disorders, cerebellar atazia, gastrointestinal tract disorders, negative symptoms of schizophrenia, premenstrual syndrome, Fibromyalgia Syndrome, stress incontinence, Tourette syndrome, trichotillomania, kleptomania, male impotence, cancer, chronic paroxysmal hemicrania and headache in a mammal, preferably a human, comprising (i) a γ-aminobutyric acid modulator or a pharmaceutically acceptable salt thereof,
(ii) a 5-HT_{1B} receptor antagonist or a pharmaceutically acceptable salt thereof,
   wherein the 5-HT_{1B} receptor antagonist is selected from the group consisting of (A) a compound of the formula I as described in the specification and (B) a compound of the formula II as described in the specification, and optionally (iii) a pharmaceutically acceptable carrier.

### BACKGROUND OF THE INVENTION

The present invention relates to pharmaceutical compositions containing γ-aminobutyric acid modulators or pharmaceutically acceptable salts thereof and 5-HT_{1B} receptor antagonists or pharmaceutically acceptable salts thereof, and to their medicinal use for treating disorders associated with the central nervous system.

U.S. Pat. Nos. 6,462,048, 6,258,953, 6,380,186, and 6,323,229, and U.S. patent Publication Nos. 2002/0091119 and 2003/0083337 describe certain aralkyl and aralkylidene heterocyclic lactams and imides that are 5-HT_{1B} receptor antagonists and that are used in the compositions of the present invention. Other 5-HT₁ receptor antagonists are described in European Patent Publications 701,819, 434,561 and 343,050, PCT publications WO 94/21619, WO 95/31988, and WO 96/00720, Glennon et al., “5-HT_{1D} Serotonin Receptors”, Clinical Drug Res. Dev., 22, 25-36 (1991), and G Maura et al., J. Neurochem, 66 (1), 203-209 (1996). These references describe 5-HT₁ receptor antagonists, including 5-HT_{1B} receptor antagonists, as useful in the treatment of, for example, migraine, depression, obsessive compulsive disorder, post-traumatic stress disorder (PTSD), and eating disorders, as well as other disorders associated with the central nervous system.

γ-Aminobutyric acid (GABA) is the major inhibitory neurotransmitter in the patient in the central nervous system (CNS). GABA receptors can be found in 60-80% of CNS neurons. Allosteric facilitation of GABA receptors occurs at several distinct sites; the compounds which bind to these receptor sites have been used as sedatives and anxiolytics.

GABA modulators have also been disclosed to be useful in antiseizure therapy for central nervous system disorders such as epilepsy, Huntington's chorea, cerebral ischemia, Parkinson's disease, tardive dyskinesia and spasticity. GABA modulators have also been disclosed to be useful antidepressants, anxiolytics and antipsychotics.

Commonly assigned U.S. Pat. No. 4,024,175, which is hereby incorporated by reference, discloses that GABA modulators have utility in the treatment of treatment-resistant anxiety, psychotic disorders and conditions, and mood disorders and conditions.

GABA modulators or agonists well known in the art include muscimol, progabide, riluzole, baclofen, gabapentin (Neurontin®), vigabatrin, valproic acid, (Depakene®, Depakote®) tiagabine (Gabitril®), lamotrigine (Lamictal®), pregabalin, topiramate (Topamax®) and analogs, derivatives, prodrugs and pharmaceutically acceptable salts thereof.

WO 01/24791 discloses the synergistic combinations of NK1 antagonists and GABA analogs and modulators.

The term “GABA”, where used in the description and the appendant claims, is synonymous with the term “gamma-aminobutyric acid.” These terms are used interchangeably throughout the description and claims.

### SUMMARY OF THE INVENTION

The present invention relates to a pharmaceutical composition for treating, for example, a disorder or condition selected from the group consisting of hypertension, depression, generalized anxiety disorder, phobias, posttraumatic stress disorder, avoidant personality disorder, sexual dysfunction, eating disorders, obesity, chemical dependencies, cluster headache, migraine, pain, Alzheimer's disease, obsessive-compulsive disorder, panic disorder, memory disorders, Parkinson's diseases, endocrine disorders, cerebellar ataxia, gastrointestinal tract disorders, negative symptoms of schizophrenia, premenstrual syndrome, Fibromyalgia Syndrome, stress incontinence, Tourette syndrome, trichotillomania, kleptomania, male impotence, cancer, chronic paroxysmal hemicrania and headache in a mammal, preferably a human, comprising:
(i) a γ-aminobutyric acid modulator or a pharmaceutically acceptable salt thereof,
(ii) a 5-HT_{1B} receptor antagonist or a pharmaceutically acceptable salt thereof, wherein the 5-HT_{1B} receptor antagonist is selected from the group consisting of
(A) a compound of the formula I 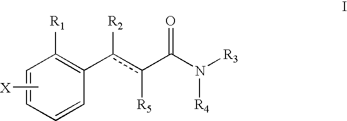
   wherein, in formula l:
R¹ is a group of the formula G¹, G², G³, G⁴, G⁵, G⁶ or G⁷ depicted below, 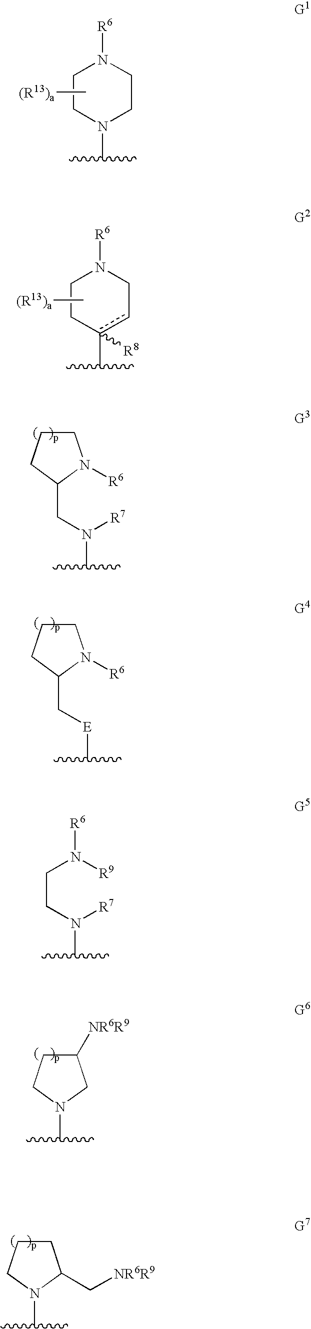
a is zero to eight;
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G² respectively, having an available bonding site, or to a ring carbon of R⁶ having an available bonding site;
E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —SOₜ(C₁-C₆)alkyl wherein t is zero, one or two, —CO₂R¹⁰ or —CONR¹¹R¹²,
R² is hydrogen, (C₁-C₄)alkyl, phenyl or naphthyl, wherein said phenyl or naphthyl is optionally substituted with one or more substituents independently selected from the group consisting of chloro, fluoro, bromo, iodo, (C₁-C₆)alkyl, (C₁-C₆)alkoxy, trifluoromethyl, cyano and —SOₖ(C₁-C₆)alkyl wherein k is zero, one or two;
R³ is —(CH₂)ₘB, wherein m is zero, one, two or three and B is hydrogen, phenyl, naphthyl or a 5 or 6 membered heteroaryl group containing from one to four hetero-atoms in the ring, and wherein each of the foregoing phenyl, naphthyl and heteroaryl groups is optionally substituted with one or more substituents independently selected from the group consisting of chloro, fluoro, bromo, iodo, (C₁-C₆)alkyl, (C₁-C₆)alkoxy, (C₁-C₆) alkoxy-(C₁-C₆)alkyl-, trifluoromethyl, trifluoromethoxy, cyano, hydroxy, —COOH and —SOₙ(C₁-C₆)alkyl wherein n is zero, one or two;
R⁴ is (C₁-C₆)alkyl or C₆-C₁₀ aryl;
or R³ and R⁴ may optionally be taken together with the nitrogen to which they are attached to form a five to seven membered heteroalkyl ring, wherein any two of the carbon atoms of said heteroalkyl ring is optionally replaced with a heteroatom selected from the group consisting of nitrogen, oxygen or sulfur (e.g., pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1-yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, piperazine, etc.); wherein said heteroalkyl ring may optionally be substituted by aryl or heteroaryl (e.g., furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indazolyl, isoquinolyl, quinolyl, phthalazinyl, quinoxalinyl, quinazolinyl, benzoxazinyl; etc.);
R⁵ is hydrogen, (C₁-C₆)alkyl or aryl, wherein aryl is selected from the group consisting of phenyl or naphthyl, wherein any of said aryl is optionally independently substituted on any available bonding site by any of the radicals of X;
or R⁵ and R⁴ taken together form a divalent group —Yₙ₂—;
Y is selected from the group consisting of (a) CR⁴R⁵, wherein R⁴ and R⁵ are independently selected from hydrogen, (C₁-C₆)alkyl and trifluoromethyl; (b) a phenylene, naphthylene or a 5 or 6 membered heteroarylene ring comprising from one to four hetero-atoms in the heteroarylene ring, and wherein each of the foregoing phenylene, naphthylene and heteroarylene rings may optionally be substituted with one or more substituents independently selected from the group consisting of chloro, fluoro, bromo, iodo, (C₁-C₆)alkyl, (C₁-C₆)alkoxy, (C₁-C₆) alkoxy-(C₁-C₆)alkyl-, trifluoromethyl, trifluoromethoxy, cyano, hydroxy, —COOH and —SOₙ(C₁-C₆)alkyl wherein n is zero, one or two, wherein two adjacent ring atoms of ring Y are also ring atoms of ring A; and (c) an optionally substituted (C₁-C₄) heteroalkyl bridge that, together with the atoms to which it is attached, forms a five to seven membered heterocycle containing two to four heteroatoms selected from the group consisting of 1,3-oxazolidin-4-on-5-yl, 1,3-oxazolidin-2,4-dion-5-yl, 4,5-dihydro-1,2-oxazolidin-3-on-4-yl, 1,3-thiazolidin-4-on-5-yl, 1,3-thiazolidin-2,4-dion-5-yl, 1,3-pyrazolidin-4-on-5-yl, 1,3-imidazolidin-2,4-dion-5-yl, 1,2-pyrazolidin-3-on-4-yl, 1,2-thiazolidin-1,1,3-trion-4-yl, 1,2-thiazolidin-3-on-4-yl, tetrahydro-1,2-oxazin-3-on-4-yl, tetrahydro-1,3-oxazin-4-on-5-yl, tetrahydro-1,3-oxazin-2,4-dion-5-yl, morpholin-3-on-2-yl, morpholin-3,5-dion-2-yl, 2,3-dihydro-1,4-oxazin-3-on-2-yl, tetrahydro-1,3-thiazin-4-on-5-yl, tetrahydro-1,3-thiazin-2,4-dion-5-yl, tetrahydro-1,2-thiazin-3-on-4-yl, thiomorpholin-3-on-2-yl, thiomorpholin-3,5-dion-2-yl, 2,3-dihydro-1,4-thiazin-3-on-2-yl, hexahydro-1,2-diazin-3-on-4-yl, 4,5-dihydro-2H-pyridazin-3-on-4-yl, hexahydro-1,3-diazin-4-on-5-yl, hexahydro-1,3-diazin-2,4-dion-5-yl, piperazin-2-on-3-yl, piperazin-2,6-dion-3-yl, tetrahydro-1,3,4-thiadiazin-5-on-6-yl, 5,6-dihydro-1,3,4-thiadiazin-5-on-6-yl, 1,3,4-oxadiazin-5-on-6-yl, 5,6-dihydro-1,2,4-oxadiazin-5-on-6-yl, tetrahydro-1,2,4-oxadiazin-5-on-6-yl, 1,2,4-triazin-5-on-6-yl, tetrahydro-1,2,4-oxadiazin-5-on-6-yl, 5,6-dihydro-1,2,4-oxadiazin-5-on-6-yl, 1,2,4-oxadiazin-3,5-dion-6-yl, 1,2,4-trazin-6-on-5-yl, hexahydro-1,2-oxazepin-3-on-2-yl, hexahydro-1,3-oxazepin-4-on-5-yl, hexahydro-1,4-oxazepin-3-on-2-yl, hexahydro-1,4-oxazepin-3,5-dion-2-yl, hexahydro-1,4-oxazepin-3,5-dion-6-yl, 2,3,5,6-tetrahydro-1,4-oxazepin-5,7-dion-6-yl, hexahydro-1,4-oxazepin-5-on-6-yl, hexahydro-1,3-oxazepin-2,4-dion-5-yl, hexahydro-1,2-thiazepin-3-on-4-yl, hexahydro-1,4-thiazepin-3-on-2-yl, 2,3,4,5-tetrahydro-1,4-thiazepin-3-on-2-yl, hexahydro-1,4-thiazepin-3, 5-dion-2-yl, hexahydro-1,4-thiazepin-3,5-dion-6-yl, 2,3,6,7-tetrahydro-1,4-thiazepin-5-on-6-yl, 6,7-dihydro-1,4-thiazepin-5-on-6-yl, hexahydro-1,3-thiazepin-2,4-dion-5-yl, hexahydro-1,2-diazepin-3-on-4-yl, hexahydro-1,3-diazepin-2,4-dion-5-yl, hexahydro-1,4-diazepin-2-on-3-yl, hexahydro-1,4-diazepin-5-on-6-yl, hexahydro-1,4-diazepin-5,7-dion-6-yl, hexahydro-1,3,5-thiadiazepin-3-on-7-yl, 4,5,6,7-tetrahydro-1,3,5-thiadiazepin-6-on-7-yl, and 2,3,5,6-tetrahydro-1,2,4-triazepin-3,5-dion-7-yl; wherein the substituents on any of the carbon atoms capable of supporting an additional bond, of said (C₁-C₄) heteroalkyl bridge, are chloro, fluoro, (C₁-C₆)alkyl, (C₁-C₆)alkoxy, trifluoromethyl or cyano; wherein the substituents on any of the nitrogen atoms capable of supporting an additional bond, of said (C₁-C₄) heteroalkyl bridge, are (C₁-C₆)alkyl or trifluoromethyl,
n2 is one, two, three or four, with the proviso that n2 is one when Y is not CR⁴R⁵;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a C₂-C₄ alkylene chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
and p is one, two, or three;
each of R¹⁰, R¹¹ and R¹² is selected, independently, from the groups set forth in the definition of R²; or R¹¹ and R¹², together with the nitrogen to which they are attached, form a 5 to 7 membered heteroalkyl ring that may contain, in addition to the nitrogen atom to which R¹¹ and R¹² are attached, from zero to four heteroatoms selected from the group consisting of nitrogen, sulfur and oxygen, and
the broken lines indicate optional double bonds, with the proviso that when the broken line in G² is a double bond, R⁸ is absent;
(B)
a compound of the formula II 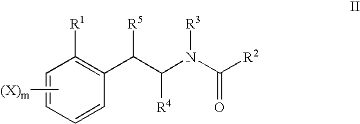
   wherein in Formula II,
R¹ is a group of the formula G¹, G², G³, G⁴, G⁸ or G^{6.} wherein G¹, G², G³, G⁴, and G⁶ are each defined as for formula I, and G⁸ is depicted below 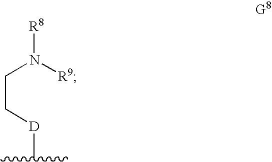
m is 0, 1, 2, 3 or 4;
D is oxygen, sulfur, SO, SO₂, or NR⁷;
a is zero to eight;
p is 1, 2 or 3;
E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —S(O)ₜ(C₁-C₆)alkyl wherein t is 0, 1 or 2, —CO₂R¹⁰ or —CONR¹¹R¹²;
R² is —(CH₂)_{y}B, wherein y is 0, 1, 2 or 3, and B is hydrogen, phenyl, naphthyl or a 5 or 6 membered heteroaryl group containing from one to four heteroatoms in the ring, and wherein each of the foregoing phenyl, naphthyl and heteroaryl groups may optionally be substituted with one or more substituents independently selected from chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is 0, 1 or 2;
R³ and R⁴ are each independently hydrogen, (C₁-C₄)alkyl or —(CH₂)_{q}-J wherein q is 0, 1, 2 or 3, and J is phenyl or naphthyl, wherein said phenyl or naphthyl may be optionally substituted with one to three substituents independently selected from the group consisting of chloro, fluoro, bromo, iodo, (C₁-C₆)alkyl, (C₁-C₆)alkoxy, trifluoromethyl, cyano and —S(O)ₖ(C₁-C₆)alkyl wherein k is 0, 1 or 2;
R⁵ is hydrogen or (C₁-C₃)alkyl;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q2}—, wherein the heteroaryl moiety is selected from the group consisting of pyridyl, pyrimidyl, benzoxazolyl, benzothiazolyl, benzisoxazolyl and benzisothiazolyl and q2 is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a 2 to 4 carbon chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
each of R¹⁰, R¹¹ and R¹² is selected, independently, from the groups set forth in the definition of R³; or R¹¹ and R¹², together with the nitrogen to which they are attached, form a 5 to 7 membered heteroalkyl ring that may contain, in addition to the nitrogen atom to which R¹¹ and R¹² are attached, from zero to four heteroatoms selected from the group consisting of nitrogen, sulfur and oxygen, and
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
with the proviso that when B is hydrogen, t is not zero; and
with the proviso that when the broken line in formula G² is a double bond, R⁸ is absent;
and optionally
(iii) a pharmaceutically acceptable carrier.

The present invention also relates to:
a pharmaceutical composition for treating, for example, a disorder or condition that can be treated by enhancing serotonergic neurotransmission in a mammal, preferably a human, comprising components (i), (ii) and optionally (iii) defined in the previous paragraphs;
a method for treating a disorder or condition as defined in the previous paragraphs in a mammal, preferably a human, comprising administering to a mammal in need of such treatment components (i) and (ii) as defined in the previous paragraphs;
a method for treating a disorder or condition that can be treated by enhancing serotonergic neurotransmission in a mammal, preferably a human, comprising administering to a mammal in need of such treatment components (i) and (ii) as defined in the previous paragraphs.
The 5-HT_{1B} receptor antagonist of the formula I or II defined herein of the compositions and the methods of the invention may be used in an amount that is a serotonin receptor antagonizing or agonizing effective amount.

In the pharmaceutical compositions and methods of the invention, components (i) and (ii) as defined in the previous paragraphs may also be combined with a 5-HT_{1A} antagonist or a pharmaceutically acceptable salt thereof, wherein the amounts of each of components (i), (ii) and the 5-HT_{1A} antagonist or a pharmaceutically acceptable salt thereof are such that the combination of components (i), (ii) and the 5-HT_{1A} antagonist or a pharmaceutically acceptable salt thereof is effective in treating a disorder or condition as defined in the previous paragraphs. For example, the method of the invention may further comprise administering a 5-HT_{1A} antagonist or a pharmaceutically acceptable salt thereof, wherein the amounts of each of components (i), (ii) and the 5-HT_{1A} antagonist or a pharmaceutically acceptable salt thereof are such that the combination of components (i), (ii) and the 5-HT_{1A} antagonist or a pharmaceutically acceptable salt thereof is effective in treating the disorder or condition.

### DETAILED DESCRIPTION OF THE INVENTION

“Enhancing serotonergic neurotransmission,” as used herein, refers to increasing or improving the neuronal process whereby serotonin is released by a pre-synaptic cell upon excitation and crosses the synapse to stimulate or inhibit the post-synaptic cell.

“Chemical dependency,” as used herein, means an abnormal craving or desire for, or an addiction to a drug. Such drugs are generally administered to the affected individual by any of a variety of means of administration, including oral, parenteral, nasal or by inhalation. Examples of chemical dependencies treatable by the methods of the present invention are dependencies on alcohol, nicotine, cocaine, amphetamine and other psychostimulants, morphine, heroin and other opioid agonists, phenobarbital and other barbiturates, and benzodiazepines such as diazepam and others. “Treating a chemical dependency,” as used herein, means reducing or alleviating such dependency.

A “unit dosage form” as used herein is any form that contains a unit dose of the γ-aminobutyric acid modulator or a pharmaceutically acceptable salt thereof, of the compound of formula I or formula II or a pharmaceutically acceptable salt thereof, or of the γ-aminobutyric acid modulator or pharmaceutically acceptable salt thereof and the compound of formula I or formula II or pharmaceutically acceptable salt thereof. A unit dosage form may be, for example, a tablet or a capsule. A unit dose may be an amount which may be predetermined, for example, by a physician.

The term “GABA modulator” as used herein refers to a compound that either is structurally related to the neurotransmitter GABA but does not interact with the GABA receptor (e.g. gabapentin), or interacts with the GABA receptors, or is converted metabolically into GABA or a GABA modulator; or is an inhibitor of GABA uptake or degradation; or is a GABA receptor subtype-selective antagonist and/or agonist. This definition includes pharmaceutically acceptable salts, prodrugs or pharmaceutically acceptable salts of said prodrugs.

Examples of the disorders or conditions which may be treated by the methods, compositions and kits of this invention are as follows:
depression, including depression in cancer patients, depression in Parkinson's patients, Postmyocardial Infarction depression, Subsyndromal Symptomatic depression, depression in infertile women, pediatric depression, major depression, single episode depression, recurrent depression, child abuse induced depression, post partum depression, DSM-IV major depression, treatment-refractory major depression, bipolar depression BP I, bipolar depression BP II, depression in patients with human immunodeficiency virus (HIV), severe depression, psychotic depression, post-stroke depression, neuropathic pain, manic depressive illness, including manic depressive illness with mixed episodes and manic depressive illness with depressive episodes, seasonal affective disorder, and major depression with dysthymia.
phobias, including agoraphobia, social phobia and simple phobias;
sexual dysfunction, including premature ejaculation;
eating disorders, including anorexia nervosa and bulimia nervosa;
chemical dependencies, including addictions to alcohol, cocaine, heroin, phenolbarbitol, nicotine and benzodiazepines;
memory disorders, including dementia, amnestic disorders, and age-related cognitive decline (ARCD);
Parkinson's diseases, including dementia in Parkinson's disease, neuroleptic-induced parkinsonism and tardive dyskinesias;
endocrine disorders, including hyperprolactinaemia;
vasospasm, including a vasospasm in the cerebral vasculature;
gastrointestinal tract disorders, including gastrointestinal tract disorders involving changes in motility and secretion;
cancer, including small cell lung carcinoma;
headache, including headache associated with vascular disorders.

Preferred disorders or conditions that may be treated by the methods, compositions and kits of this invention are migraine, depression, obsessive compulsive disorder, post-traumatic stress disorder (PTSD), and eating disorders.

As used herein, “mammal” means any member of the class Mammalia. As an example, the mammal in need of the treatment may be a human. As another example, the mammal in need of the treatment may be a mammal other than a human.

The methods of this invention also encompass treating the diseases or conditions described herein by the co-administration of two separate pharmaceutical compositions. In this latter embodiment, a first composition comprises a y-aminobutyric acid modulator, and a second composition comprises a 5-HT_{1B} receptor antagonist of the formula I or II. These first and second compositions are preferably co-administered either simultaneously, or in a specifically timed manner.

A prodrug of the γ-aminobutyric acid modulator, of the 5-HT_{1B} receptor antagonist of the formula I or II, or of both the γ-aminobutyric acid modulator and the 5-HT_{1B} receptor antagonist also may be used in the composition and method of the invention. The term “prodrug” refers to compounds that are drug precursors which, following administration, release the drug in vivo via some chemical or physiological process (e.g., a prodrug on being brought to the physiological pH is converted to the desired drug form). A prodrug of any or all of the γ-aminobutyric acid modulators or the 5-HT_{1B} receptor antagonists may be used in the methods, kits, and compositions of the instant invention. In general, prodrugs are functional derivatives of these compounds which are readily convertible in vivo. Conventional procedures for the selection and preparation of suitable prodrug derivatives are described, for example, in Design of Prodrugs, ed. H. Bundgaard, Elsevier, 1985 and can be achieved using methods well known to those skilled in the art. All such prodrugs are within the scope of the combinations, pharmaceutical compositions, methods and kits of this invention.

Upon cleavage, exemplary prodrugs release the corresponding free acid (where applicable), and such hydrolyzable ester-forming residues of the prodrugs of this invention include but are not limited to carboxylic acid substituents wherein the free hydrogen is replaced by (C₁-C₄)alkyl, (C₂-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as N,N-dimethylaminoethyl), carbamoyl-(C₁-C₂)alkyl, N,N-di(C₁-C₂)-alkylcarbamoyl-(C₁-C₂)alkyl, piperidino-, pyrrolidino-, or morpholino(C₂-C₃)alkyl, and the like.

The present invention also relates to the pharmaceutically acceptable acid addition salts of compounds of the formula I or formula II. The acids which are used to prepare the pharmaceutically acceptable acid addition salts of the aforementioned base compounds of this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3-naphthoate)]salts.

The invention also relates to base addition salts of formula I or formula II. The chemical bases that may be used as reagents to prepare pharmaceutically acceptable base salts of those compounds of formula I or formula II that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

The compounds of this invention include all stereoisomers (e.g., cis and trans isomers) and all optical isomers of compounds of the formula I or formula II (e.g., R and S enantiomers), as well as racemic, diastereomeric and other mixtures of such isomers.

The compounds of this invention may contain C═C double bonds. When such bonds are present, the compounds of the invention exist as cis and trans configurations and as mixtures thereof.

Unless otherwise indicated, the alkyl and alkenyl groups referred to herein, as well as the alkyl moieties of other groups referred to herein (e.g., alkoxy), may be linear or branched, and they may also be cyclic (e.g., cyclopropyl, cyclobutyl, cyclopentyl, or cyclohexyl) or be linear or branched and contain cyclic moieties. Unless otherwise indicated, halogen includes fluorine, chlorine, bromine, and iodine.

The term “a 5 or 6 membered heteroaryl group containing from one to four heteroatoms in the ring”, as used herein, unless otherwise indicated, includes but is not limited to furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indazolyl, isoquinolyl, quinolyl, phthalazinyl, quinoxalinyl, quinazolinyl or benzoxazinyl.

The term “a 5 to 7 membered heteroalkyl ring that may contain from one to four heteroatoms selected from nitrogen, sulfur and oxygen”, as used herein, unless otherwise indicated, includes but is not limited to pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1-yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, piperazine.

The following are more specific embodiments of groups G¹ and G² of the compound of formula I: 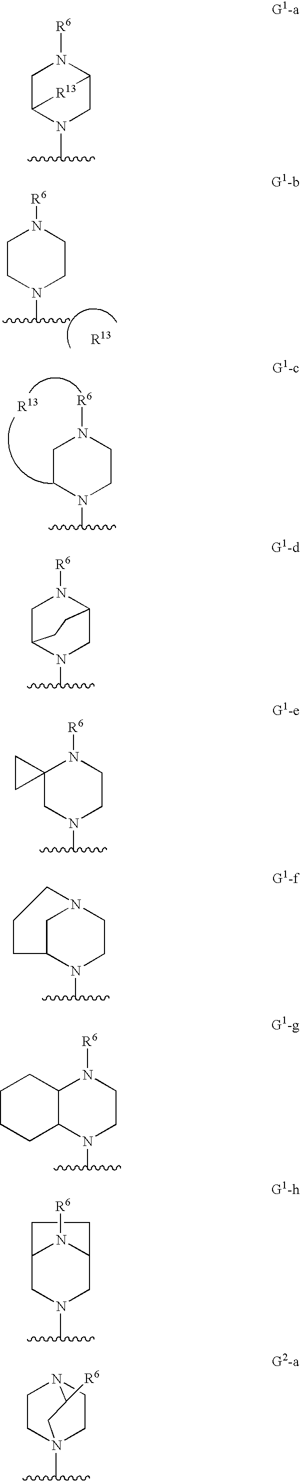
wherein each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site.

Preferred compounds of the formula I include those wherein R¹ is 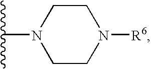
R⁶ is (C₁-C₆)alkyl, such as methyl, and R² is hydrogen.

Other preferred compounds of formula I include those wherein R³ is hydrogen, phenyl or benzyl optionally substituted by chloro, fluoro, bromo, iodo, (C₁-C₆)alkyl or trifluoromethyl.

Other preferred compounds of formula I include those wherein R⁴ is hydrogen or (C₁-C₆)alkyl, such as methyl.

More preferred compounds of formula I include those wherein R¹ is 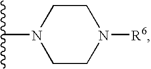
R⁶ is (C₁-C₆)alkyl and R² is hydrogen; R³ is phenyl or benzyl optionally substituted by chloro, fluoro, bromo, iodo, (C₁-C₆)alkyl or trifluoromethyl; and R⁴ is hydrogen or (C₁-C₆)alkyl.

Preferred compounds of the formula I also include those wherein Y, together with the atoms to which it is attached, forms an optionally substituted five to seven membered heterocycle selected from the group consisting of 1,3-thiazolidin-2,4-dion-5-yl, 1,3-imidazolidin-2,4-dion-5-yl, thiomorpholin-3-on-2-yl or morpholin-3-on-2-yl.

Preferred compounds of the formula I also include those wherein R³ is phenyl or —(CH₂)-phenyl, wherein said phenyl groups are optionally substituted with one or more substituents independently selected from the group consisting of chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n in —SOₙ(C₁-C₆)alkyl is zero, one or two.

Preferred compounds of the formula I also include those wherein R⁵ is hydrogen or methyl.

Preferred compounds of the formula I also include those wherein X is hydrogen, fluoro or chloro, preferably wherein X is hydrogen.

Preferred compounds of the formula I also include those wherein R⁴ and R⁵, together with the nitrogen to which they are attached, form a 5 to 7 membered heteroalkyl ring that is selected from the group consisting of pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1-yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, and piperazine.

Preferred compounds of the formula I also include those wherein m is 0 or 1.

Preferred compounds of the formula II include those wherein R¹ is 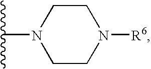
R⁶ is (C₁-C₆)alkyl and R³ is hydrogen.

Other preferred compounds of formula II include those wherein R² is phenyl or benzyl optionally substituted by chloro, fluoro, bromo, iodo, (C₁-C₆)alkyl or trifluoromethyl.

Other preferred compounds of formula II include those wherein R⁴ is hydrogen or (C₁-C₆)alkyl.

More preferred compounds of formula II include those wherein R¹ is 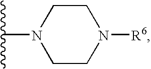
R⁶ is (C₁-C₆)alkyl and R³ is hydrogen; R² is phenyl or benzyl optionally substituted by chloro, fluoro, bromo, iodo, (C₁-C₆)alkyl or trifluoromethyl; and R⁴ is hydrogen or (C₁-C₆)alkyl.

Preferred examples of compounds of component (ii) include:
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thiomorpholin-3-one;
2-[2-(4-methylpiperazin-1-yl )-benzylidene]-thiomorpholin-3-one;
4-(3,4-dichlorophenyl)-2-[2-fluoro-6-(4-methylpiperazin-1-yl )-benzylidene]-thiomorpholin-3-one;
4-(3,4-dichlorophenyl)-2-[2-(4-methylpiperazin-1-yl)-benzylidene]-thiomorpholin-3-one;
4-(3,4-dichlorophenyl)-2-[2-(4-methylpiperazin-1-yl)-benzyl]-thiomorpholin-3-one;
4-methyl-2-[2-(4-methylpiperazin-1-yl)-benzylidene]-thiomorpholin-3-one;
4-(3,4-dichlorophenyl)-2-(2-piperazin-1-ylbenzylidene)-thiomorpholin-3-one;
4-(3,4-dichlorophenyl)-2-[2-(4-methyl-(4-piperazin-1-yl)-benzylidene]-1-oxo-thiomorpholin-3-one;
4-(3,4-dichlorophenyl)-2-[2-(4-methyl-4-oxy-piperazin-1-yl)-benzylidene]-thiomorpholin-3-one;
10-[4(3,4-dichlorophenyl)-3-oxo-thiomorpholin-2-yl]-2-methyl-3,42-dihydro-pyrazino[1,2-a]indol-2-ium;
4-Benzyl-2-[2-(4-methylpiperazin-1-yl)-benzylidene]-1,1-dioxothiomorpholin-3-one;
4-(3,4-Dichlorophenyl)-2-[3-fluoro-2-(4-methylpiperazin-1-yl)-benzylidene]-thiomorpholin-3-one;
4-(3,4-Dichlorophenyl)-2-[5-fluoro-2-(4-methylpiperazin-1-yl)-benzylidene]-thiomorpholin-3-one;
4-(3,4-Dichlorophenyl)-2-[2-(4-methylpiperazin-1-yl)-5-trifluoromethyl-benzylidene]-thiomorpholin-3-one;
4-(3,4-Dichlorophenyl)-2-{2-[4-(2-methoxyethyl )piperazin-1-yl]-benzylidene}thiomorpholin-3-one;
4-(3,4-Dichlorophenyl)-2-[2-(4-isopropylpiperazin-1-yl)-benzylidene]-thiomorpholin-3-one;
4-(3,4-Dichlorophenyl)-2-[2-(4-ethylpiperazin-1-yl)-benzylidene]-thiomorpholin-3-one;
4-(4-Chlorophenyl)-2-[2-(4-methylpiperazin-1-yl)-benzylidene]-thiomorpholin-3-one;
4-(3-Chlorophenyl)-2-[2-(4-methylpiparazin-1-yl)-benzylidene]-thiomorpholin-3-one;
2-[2-Chloro-6-(4-methylpiperazin-1-yl)-benzylidene]-4-(3,4-dichlorophenyl)-thiomorpholin-3-one;
4-(3,4-Dichlorophenyl)-2-[2-(4-methylpiperazin-1-yl)-4-trifluoromethyl-benzylidene]-thiomorpholin-3-one;
4-(3,4-Dichlorophenyl)-2-[2-(4-methylpiperazin-1-yl)-benzylidene]-1-oxo-thiomorpholin-3-one;
4-(3,4-Dichlorophenyl)-2-(5-fluoro-2-piperazin-1-yl-benzylidene)-thiomorpholin-3-one;
4-(3,4-Dichlorophenyl)-2-[3,6-difluoro-2-(4-methylpiperazin-1-yl)-benzylidene]-thiomorpholin-3-one;
4-(3,4-Dichlorophenyl)-2-[2-(3,5-dimethylpiperazin-1-yl)-benzylidene]-thiomorpholin-3-one;
4-Phenyl-2-[2-(3,4,5-trimethylpiperazin-1-yl)-benzylidene]-thiomorpholin-3-one;
2-[5-Fluoro-2-(4-methylpiperazin-1-yl)-benzylidene]-4-phenyl-thiomorpholin-3-one;
4-Benzo[1,3]dioxol-5-yl-2-[2-(3,5-dimethylpiperazin-1-yl)-benzylidene]-thiomorpholin-3-one;
2-[2-(4-tert-Butylpiperazin-1-yl)-benzylidene]-4-(3,4-dichlorophenyl)-thiomorpholin-3-one;
3-[4-(3,4-Dichlorophenyl )-3-oxo-thiomorpholin-2-ylidenemethyl]-6-dimethylamino-2-(4-methylpiperazin-1-yl)-benzonitrile;
4-(3,4-Dichlorophenyl)-2-[2-(3,4,5-trimethylpiperazin-1-yl)-benzylidene]-thiomorpholin-3-one;
4-(3,4-Dichlorophenyl)-2-[5-methyl-2-(4-methylpiperazin-1-yl)-benzylidene]-thiomorpholin-3-one;
2-[4-Chloro-2-(4-methylpiperazin-1-yl)-benzylidene]-4-(3,4-dichlorophenyl)-thiomorpholin-3one;
4-(3,4-Difluorophenyl)-2-[2-(3,5-dimethylpiperazin-1-yl)-benzylidene]-thiomorpholin-3-one;
4-(2,4-Difluorophenyl)-2-[2-(3,5-dimethylpiperazin-1-yl)-benzylidene]-thiomorpholin-3-one;
2-(4-Bromo-2-(4-methylpiperazin-1-yl)-benzylidene]-4-(3,4-dichlorophenyl)-thiomorpholin-3one;
4-(3,4-Dichlorophenyl)-2-[2-(1-methylpyrrolidin-2-ylmethoxy)-benzylidene]-thiomorpholin-3-one;
4-(3,5-Dichlorophenyl)-2-[2-(3,5-dimethylpiperazin-1-yl)-benzylidene]-thiomorpholin-3-one;
4-(3,4-Difluorophenyl)-2-[2-(3,4,5-trimethylpiperazin-1-yl)-benzylidene]-thiomorpholin-3-one;
4-(3,4-Dichlorophenyl)-2-[2-(octahydropyrido[1,2-a]pyrazin-2-yl)-benzylidene]-thiomorpholin-3-one;
2-[2-(4-Cyclopropylpiperazin-1-yl)-benzylidene]-4-pyridin-3-yl-thiomorpholin-3-one;
2-[2-(4-Cyclopropylpiperazin-1-yl)-benzylidene]-4-(3,4-difluorophenyl)-thiomorpholin-3-one;
2-[2-(4-Cyclopropylpiperazin-1-yl)-benzylidene]-4-(3,5-dichlorophenyl)-thiomorpholin-3-one;
4-(3,4-Difluorophenyl)-2-[2-(2,5-dimethylpiperazin-1-yl)-benzylidene]-thiomorpholin-3-one;
4-(3,5-Dichlorophenyl)-2-[2-(2,5-dimethylpiperazin-1-yl)-benzylidene]-thiomorpholin-3-one;
4-(3,4-Dichlorophenyl)-2-[2-(3-methylaminopyrrolidin-1-yl)-benzylidene]-thiomorpholin-3-one;
4-(3,4-Difluorophenyl)-2-[2-(2,4,5-trimethylpiperazin-1-yl)-benzylidene]-thiomorpholin-3-one;
4-Benzo[1,3]dioxol-5-yl-2-[2-(4-cyclopropylpiperazin-1-yl)-benzylidene]-thiomorpholin-3-one;
2-[2-(3,5-Dimethylpiperazin-1-yl)-benzylidene]-4-(4-fluorophenyl)-thiomorpholin-3-one;
4-Benzo[1,3]dioxol-5-yl-2-[2-(2,5-dimethylpiperazin-1-yl)-benzylidene]-thiomorpholin-3-one;
2-[2-(3,5-Dimethylpiperazin-1-yl)-benzylidene]-4-phenylthiomorpholin-3-one;
4-(3,4-Dichlorophenyl)-2-[2-(4-methylpiperazin-1-yl)-benzylidene]-thiomorpholin-3-one;
4-(3,4-Dichlorophenyl)-2-[2-(3-dimethylaminopyrrolidin-1-yl)-benzylidene]-thiomorpholin-3-one;
4-(3,4-Dichlorophenyl)-2-[2-(3-dimethylaminopyrrolidin-1-yl)-benzylidene]-thiomorpholin-3-one;
4-(3,4-Dichlorophenyl)-2-[2-(4-methyl-[1,4]diazepan-1-yl)-benzylidene]-thiomorpholin-3-one;
4-(3,4-Dichlorophenyl)-2-[2-(2,4,6-trimethylpiperazin-1-yl)-benzylidene]-thiomorpholin-3-one;
2-[2-(4-Cyclopropylpiperazin-1-yl)-benzylidene]-4-(3,4-dichlorophenyl)-thio morpholin-3-one;
the (−)-enantiomer of a compound of formula 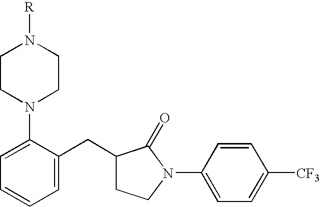
   and pharmaceutically acceptable salts thereof; wherein R is H or CH₃;
a compound of formula 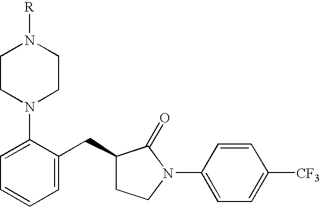
   and pharmaceutically acceptable salts thereof; wherein R is H or CH₃—;
(−)-3(S)-[[2-(4-methyl-1-piperazinyl)phenyl]methyl]-1-[4-(trifluoromethyl)phenyl]-2-pyrrolidinone;
an enantiomeric mixture of (−)-3(S)-[[2-(4-methyl-1-piperazinyl)phenyl]methyl]-1-[4-(trifluoromethyl)phenyl]-2-pyrrolidinone; and (+)-3(R)-[[2-(4-methyl-1-piperazinyl)phenyl]methyl]-1-[4-(trifluoromethyl)phenyl]-2-pyrrolidinone, or pharmaceutically acceptable salts thereof; wherein the ratio of the 3(S)-enantiomer to the (R)-enantiomer is in excess of 2:1, 5:1 or 99:1;
a compound of formula III 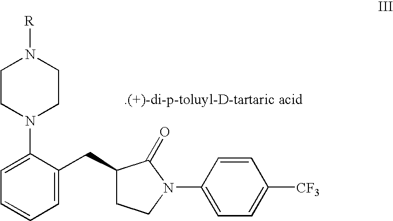
   wherein R is H or CH₃;
3,4-Dichloro-N-(2-[2-(4-methylpiperazin-1-yl)-phenyl]-ethyl)-benzamide;
4-Fluoro-N-(2-[2-(4-methylpiperazin-1-yl)-phenyl]-ethyl)-benzamide;
N-(2-[2-(4-methylpiperazin-1-yl)-phenyl]-ethyl)-benzamide;
3,4-Dichloro-N-(1-methyl-2-[2-(4-methylpiperazin-1-yl)-phenyl]-ethyl)-benzamide;
3,4-Dichloro-N-(1-methyl-2-[2-(4-methylpiperazin-1-yl)-phenyl]-propyl)-benzamide;
3,4-Dichloro-N-methyl-N-(2-[2-(4-methylpiperazin-1-yl)-phenyl]-ethyl)-benzamide;
N-Benzyl-N-(2-[2-(4-methylpiperazin-1-yl)-phenyl]-ethyl)-benzamide;
N-(4-chlorobenzyl)-N-(2-[2-(4-methylpiperazin-1-yl)-phenyl]-ethyl)-benzamide;
3,4-Dichloro-N-(2-{2-[methyl-(1-methylpyrolidin-2-ylmethyl)-amino]-phenyl}-ethyl)-benzamide;
3,4-Dichloro-N-{2-[2-(1-methyl-octahydro-pyrrolo[2,3-c]pyridin-6-yl)-phenyl]-ethyl}-benzamide;
3,4-Dichloro-N-{2-[2-(hexahydro-pyrrolo[1,2-a]pyrazin-2-yl)-phenyl]-ethyl}-benzamide;
3,4-Dichloro-N-{2-[2-(1-methylpiperidin-4-yl)-phenyl]-ethyl)-benzamide;
3,4-Dichloro-N-{2-[2-(2-dimethylaminoethoxy)-phenyl]-ethyl}-benzamide;
3,4-Dichloro-N-{2-[2-(2-dimethylamino-ethylsulfanyl)-phenyl]-ethyl}-benzamide;
3,4-Dichloro-N-{2-[2-(2-pyrrolidin-1-ylethoxy)-phenyl]-ethyl}-benzamide;
4-Chloro-N-{2-[2-(3-dimethylamino-pyrrolidin-1-yl)-phenyl]-ethyl}-benzamide;
4-Chloro-N-(2-{2-[methyl-(2-morpholin-4-yl-ethyl)-amino]-phenyl}-ethyl)-benzamide;
2-(4-Chloro-phenyl)-N-{2-[2-(4-methylpiperazin-1-yl)-phenyl]-ethyl}-acetamide;
N-{2-[2-(4-Methylpiperazin-1-yl)-phenyl]-ethyl}-N-phenylacetamide;
N-{2-[2-(4-Methylpiperazin-1-yl)-phenyl]-ethyl}-isonicotinamide;
N-{2-[2-(1-Azabicyclo[2.2.2]oct-4-yl)-phenyl]-ethyl}-N-methylbenzamide;
N-{2-[2-(1,4-Dimethylpiperidin-4-yl)-phenyl]-ethyl}-4-fluorobenzamide;
4-Fluoro-N-{2-[2-(9-methyl-3,9-diazabicyclo[3.3.1 ]non-3-yl)-phenyl]-ethyl}-benzamide;
N-(2-[2-(1,4-Diazabicyclo[3.3.1]non-4-yl)-phenyl]-ethyl}-N-methylbenzamide;
N-{1-Methyl-2-[2-(5-methyl-2,5-diazabicyclo[2.2.l]hept-2-yl)-phenyl]-ethyl}-benzamide;
2,4-Dichloro-N-methyl-N-{1-methyl-2-[2-(3-methyl-3,8-diazabicyclo[3.2.1 ]oct-8-yl)-phenyl]-ethyl}-benzamide;
N-{2-[2-(4-Methyl-octahydroquinoxalin-1-yl)-phenyl]-ethyl}-benzamide;
N-{2-[2-(1-Ethylpyrrolidin-2-ylmethoxy)-phenyl]-ethyl}-benzamide;
5-Phenyloxazole-2-carboxylic acid {2-[2-(4-methylpiperazin-1-yl)-phenyl]-ethyl}-amide;
5-Phenylthiophene-2-carboxylic acid {2-[2-(4-methylpiperazin-1-yl)-phenyl]-ethyl}-amide;
5-Methylthiophene-2-carboxylic acid {2-[2-(4-methylpiperazin-1-yl)-phenyl]-ethyl}-amide;
4-Fluoronaphthalene-1-carboxylic acid {2-[2-(4-methylpiperazin-1-yl)-phenyl]-ethyl}-amide;
5-Fluoro-1H-indole-2-carboxylic acid {2-[2-(4-methyl-piperazin-1-yl)-phenyl]-ethyl}-amide;
4-Chloro-N-{2-[2-(3,4,5-trimethylpiperazin-1-yl)-phenyl]-ethyl}-benzamide;
3,4-Dichloro-N-{2-[2-(2,4,5-trimethylpiperazin-1-yl)-phenyl]-ethyl}-benzamide; and
3,4-Dichloro-N-{2-[2-(2,4,6-trimethylpiperazin-1-yl)-phenyl]-ethyl}-benzamide.

Methods for making the 5-HT_{1B} receptor antagonists of the formula I or II described above are disclosed in the above-listed patents and published patent applications incorporated by reference herein, including, for example, U.S. Pat. Nos. 6,462,048; 6,258,953; 6,380,186; and 6,323,229; U.S. patent Publication Nos. 2002/0091119 and 2003/0083337.

The GABA modulators suitable for use in the present invention include, for example, compounds of the formula 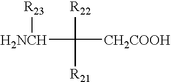
wherein R₂₁ is a straight or branched alkyl group having from 1 to 6 carbon atoms, phenyl, or cycloalky having from 3 to 6 carbon atoms; R₂₂ is hydrogen or methyl; and R₂₃ is hydrogen, methyl or carboxyl; and the pharmaceutically acceptable salts thereof.

The GABA modulators suitable for use in the present invention include, as another example, compounds of the formula 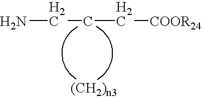
wherein R₂₄ is a hydrogen atom or a C₁-C₆ alkyl radical and n3 is 4,5, or 6; and the pharmaceutically acceptable salts thereof.

Examples of GABA modulators include, but are not limited to, muscimol, progabide, riluzole, baclofen, gabapentin (Neurontin®), vigabatrin, tiagabine (Gabitril®), lamotrigine (Lamictal®), pregabalin, topiramate (Topamax®), a prodrug thereof or a pharmaceutically acceptable salt of the GABA modulator or prodrug thereof. It will be recognized by those skilled in the art in light of this disclosure that other GABA modulators are also useful in the combinations, pharmaceutical compositions, methods and kits of this invention.

The GABA modulators disclosed herein are prepared by methods well known to those skilled in the art. Specifically, the following patents and patent applications exemplify GABA modulators which can be used in the combinations, pharmaceutical compositions, methods and kits of this invention, and refer to methods of preparing those GABA modulators: U.S. Pat. No. 3,242,190 (specifically, muscimol); U.S. Pat. No. 4,094,992 (specifically, progabide); U.S. Pat. No. 4,370,338 (specifically, riluzole); U.S. Pat. No. 3,471,548 (specifically, baclofen); U.S. Pat. No. 4,024,175 (specifically, gabapentin); U.S. Pat. No. 3,960,927 (specifically, vigabatrin); U.S. Pat. No. 5,010,090 (specifically, tiagabine); U.S. Pat. No. 4,602,017 (specifically, lamotrigine); U.S. Pat. No. 6,028,214 (specifically, pregabalin); and U.S. Pat. No. 4,513,006 (specifically, topiramate). U.S. Pat. Nos. 4,024,175 and 6,028,214 are incorporated by reference herein.

Gabapentin, 1-(aminomethyl)cyclohexane acetic acid, is an anticonvulsant indicated as adjunctive therapy in the treatment of partial seizures with or without secondary generalization in adults with epilepsy. Gabapentin and its methods of use are described in U.S. Pat. Nos. 4,024,175 and 4,087,544 incorporated herein by reference in their entirety.

It will be recognized that certain of the GABA modulators used in the pharmaceutical compositions, methods and kits of this invention contain either a free carboxylic acid or a free amine group as part of the chemical structure. Thus, this invention includes pharmaceutically acceptable salts of those carboxylic acids or amine groups.

Where the compounds of formula I or II or the GABA modulators of use in the invention have at least one asymmetric center, they may accordingly exist as enantiomers. Where the compounds according to the invention possess two or more asymmetric centers, they may additionally exist as diastereoisomers. It is to be understood that all such isomers and mixtures thereof in any proportion are encompassed within the scope of the present invention. Gabapentin may be in the form of the crystalline monohydrate as described in EP340677 which is incorporated herein by reference or the anhydrous crystalline form as described in WO 03031391.

The pharmaceutically-acceptable cationic salts of GABA modulators containing free carboxylic acids may be readily prepared by reacting the free acid form of the GABA modulator with an appropriate base, usually one equivalent, in a co-solvent. Typical bases are sodium hydroxide, sodium methoxide, sodium ethoxide, sodium hydride, potassium methoxide, magnesium hydroxide, calcium hydroxide, benzathine, choline, diethanolamine, piperazine and tromethamine. The salt is isolated by concentration to dryness or by addition of a non-solvent. In many cases, salts are preferably prepared by mixing a solution of the acid with a solution of a different salt of the cation (e.g., sodium or potassium ethylhexanoate, magnesium oleate), employing a solvent (e.g., ethyl acetate) from which the desired cationic salt precipitates, or can be otherwise isolated by concentration and/or addition of a non-solvent.

The pharmaceutically acceptable acid addition salts of GABA modulators containing free amine groups may be readily prepared by reacting the free base form of the GABA modulator with the appropriate acid. When the salt is of a monobasic acid (e.g., the hydrochloride, the hydrobromide, the p-toluenesulfonate, the acetate), the hydrogen form of a dibasic acid (e.g., the hydrogen sulfate, the succinate) or the dihydrogen form of a tribasic acid (e.g., the dihydrogen phosphate, the citrate), at least one molar equivalent and usually a molar excess of the acid is employed. However, when such salts as the sulfate, the hemisuccinate, the hydrogen phosphate or the phosphate are desired, the appropriate and exact chemical equivalents of acid will generally be used. The free base and the acid are usually combined in a co-solvent from which the desired salt precipitates, or can be otherwise isolated by concentration and/or addition of a non-solvent.

In the preferred kits of the present invention, the pharmaceutical composition comprising a γ-aminobutyric acid modulator is a pharmaceutical composition comprising one of the particularly preferred γ-aminobutyric acid modulators as defined above, and the pharmaceutical composition comprising a 5-HT_{1B} receptor antagonist is a pharmaceutical composition comprising one of the particularly preferred 5-HT_{1B} receptor antagonists as defined above.

The preferred methods of treatment of the present invention are those methods that employ a particularly preferred γ-aminobutyric acid modulator and particularly preferred 5-HT_{1B} receptor antagonist as defined above.

Also preferred are those methods that employ a particularly preferred γ-aminobutyric acid modulator and a particularly preferred 5-HT_{1B} receptor antagonist or a pharmaceutical composition(s) of the present invention, as defined above, for treating migraine, depression, obsessive compulsive disorder, post-traumatic stress disorder (PTSD), and eating disorders.

Preferably, the combinations of pharmaceutically active compounds of the present invention show a synergistic effect and/or show less side effects, as compared to the individual compounds, when treating a mammal, preferably a human. Thus, in treating a particular disease, at a specific dosage level, the combinations of pharmaceutically active compounds of the present invention show a better activity than the activity which could be expected when administering the individual compounds, less or less severe side effects than could be expected when administering the individual compounds, or a combination of a better activity and of less or less severe side effects than could be expected when administering the individual compounds.

The expression “pharmaceutically acceptable salts” includes both pharmaceutically acceptable acid addition salts and pharmaceutically acceptable cationic salts.

Compounds of the formula I and II and their pharmaceutically acceptable salts, and γ-aminobutyric acid modulators and their pharmaceutically acceptable salts are hereinafter also referred to, collectively, as “the active compounds.” Compounds of the formula I or II are useful in the treatment of hypertension, depression, generalized anxiety disorder, phobias such as agoraphobia, social phobia and simple phobias, posttraumatic stress syndrome, avoidant personality disorder, sexual dysfunction such as premature ejaculation, eating disorders such as anorexia nervosa and bulimia nervosa, obesity, chemical dependencies such as addictions to alcohol, cocaine, heroin, phenolbarbitol, nicotine and benzodiazepines, cluster headache, migraine, pain, Alzheimer's disease, obsessive-compulsive disorder, panic disorder, memory disorders such as dementia, amnestic disorders, and age-related cognitive decline (ARCD), Parkinson's diseases such as dementia in Parkinson's disease, neuroleptic-induced parkinsonism and tardive dyskinesias, endocrine disorders such as hyperprolactinaemia, vasospasm, particularly in the cerebral vasculature, cerebellar ataxia, gastrointestinal tract disorders, such as involving changes in motility and secretion, negative symptoms of schizophrenia, premenstrual syndrome, Fibromyalgia Syndrome, stress incontinence, Tourette syndrome, trichotillomania, kleptomania, male impotence, cancer such as small cell lung carcinoma, chronic paroxysmal hemicrania and headache, such as headache associated with vascular disorders. Similarly, the compositions of the present invention are useful in the treatment of the disorders or conditions listed in this paragraph.

The affinities of the compounds of the formula I for the various serotonin-1 receptors can be determined using standard radioligand binding assays as described in the literature. The 5-HT_{1A} affinity can be measured using the procedure of Hoyer et al. (Brain Res., 376, 85 (1986)). The 5-HT_{1B} affinity can be measured using the procedure of Heuring and Peroutka (J. Neurosci., 7, 894 (1987)). The activity of the compounds of the formula I or II at the 5-HT_{1B} binding site, the activity for 5-HT_{1A} binding ability, and the agonist and antagonist activities of the compounds of the formula I or II at 5-HT_{1A} and 5-HT_{1B} receptors may be determined as described in U.S. Pat. No. 6,380,186. All 5-HT_{1B} receptor antagonists that were tested exhibited IC₅₀'s less than 0.60 μM for 5-HT₁B affinity and IC₅₀'s less than 1.0 μM for 5-HT_{1A} affinity. Similarly, the activity at the 5-HT_{1B} binding site, the activity for 5-HT_{1A} binding ability, and the agonist and antagonist activities of the compositions of the present invention may be determined using the procedures described for the compounds in formula I in U.S. Pat. No. 6,380,186.

In the present invention, the 5-HT_{1B} receptor antagonists of formula I or II and the γ-aminobutyric acid modulators may also be further combined with one or more other therapeutic agents, for instance, different antidepressant agents such as tricyclic antidepressants such as amitriptyline, dothiepin, doxepin, trimipramine, butripyline, clomipramine, desipramine, imipramine, iprindole, lofepramine, nortriptyline or protriptyline, monoamine oxidase inhibitors such as isocarboxazid, phenelzine or tranylcyclopramine or monoamine reuptake inhibitors such as fluvoxamine, sertraline, fluoxetine or paroxetine, and/or with antiparkinsonian agents such as dopaminergic antiparkinsonian agents such as levodopa, preferably in combination with a peripheral decarboxylase inhibitor such as benserazide or carbidopa. It is to be understood that the present invention covers the combination of a 5-HT_{1B} receptor antagonists of formula I or II or a pharmaceutically acceptable salt thereof with a γ-aminobutyric acid modulator or a pharmaceutically acceptable salt thereof and with one or more such therapeutic agents.

The combination of the compounds of the formula I or II or the pharmaceutically acceptable salts thereof and a γ-aminobutyric acid modulator or a pharmaceutically acceptable salt thereof is also referred herein to as “the active combination.” The active combinations are useful psychotherapeutics and may be used in the treatment of disorders the treatment of which is facilitated by enhanced serotonergic neurotransmission such as hypertension, depression, generalized anxiety disorder, phobias such as agoraphobia, social phobia and simple phobias, posttraumatic stress syndrome, avoidant personality disorder, sexual dysfunction such as premature ejaculation, eating disorders such as anorexia nervosa and bulimia nervosa, obesity, chemical dependencies such as addictions to alcohol, cocaine, heroin, phenolbarbitol, nicotine and benzodiazepines, cluster headache, migraine, pain, Alzheimer's disease, obsessive-compulsive disorder, panic disorder, memory disorders such as dementia, amnestic disorders, and age-related cognitive decline (ARCD), Parkinson's diseases such as dementia in Parkinson's disease, neuroleptic-induced parkinsonism and tardive dyskinesias, endocrine disorders such as hyperprolactinaemia, vasospasm, particularly in the cerebral vasculature, cerebellar ataxia, gastrointestinal tract disorders, such as involving changes in motility and secretion, negative symptoms of schizophrenia, premenstrual syndrome, Fibromyalgia Syndrome, stress incontinence, Tourette syndrome, trichotillomania, kleptomania, male impotence, cancer such as small cell lung carcinoma, chronic paroxysmal hemicrania and headache, such as headache associated with vascular disorders.

Activity of the active combinations as antidepressants and related pharmacological properties can be determined by methods (1)-(3) below, which are described in Koe, B. et al. Journal of Pharmacology and Experimental Therapeutics, 226 (3), 686-700 (1983). Specifically, activity can be determined by studying (1) their ability to affect the efforts of mice to escape from a swim-tank (Porsolt mouse “behavior despair” test), (2) their ability to potentiate 5-hydroxytryptophan-induced behavioral symptoms in mice in vivo, and (3) their ability to block the uptake of serotonin, norepinephrine and/or dopamine by synaptosomal rat brain cells in vitro. The ability of the active combinations to counteract reserpine hypothermia in mice in vivo can be determined according to the methods described in U.S. Pat. No. 4,029,731. The activity of the active combinations as antidepressants and related pharmacological properties also can be determined by methods (4)-(8)) below. Specifically, activity can be determined by studying (4) their ability to reverse the stress-induced decrease in sucrose intake in rodents described in Papp, M. et al., European Journal of Pharmacology, 261, 141-147 (1994), (5) learned helplessness paradigm described in Martin, P et al., Life Sciences, 48, 2505-2511 (1991), (6) reversing the behavioral deficits of olfactory bulbectomized rats described in Broekkamp, C L et al., Pharmacology, Biochemistry and Behavior, 13, 643-646 (1980), (7) increasing down-regulation or desensitization of beta-adrenergic receptors described in Mishra, R. et al., Neuropharmacology, 19, 983-987 (1980), and (8) increasing extracellular levels of serotonin, norepinephrine, and/or dopamine in the prefrontal cortex of freely-moving rodents by in vivo dialysis described in Millan, M J et al., European Journal of Neuroscience, 12, 1079-1095 (2000).

Activity of the active combinations of the invention for the treatment of obsessive-compulsive disorders can be assessed (1) by measuring the attenuation of lever-pressing in the rat, described by Joel, D. and Avisar, A. in Behavioral Brain Research. 123, 77-87 (2001); (2) disruption of spontaneous alternation behavior in rat as exemplified by Yadin, E. et al in Pharmacology Biochemistry and Behavior, 40, 311-315 (1991): (3) a schedule-induced polydipsia assay in rat as disclosed in U.S. Pat. No. 5,356,910 (Kongsamut et al, Hoechst-Roussel Pharmaceuticals) issued Oct. 18, 1994. Activity of the active combinations of the invention for the treatment of sexual dysfunction can be assessed using the methods described in Hillegaart, V. and Ahlenius, S. in British Journal of Pharmacology, 125, 1733-1743 (1998).

The compositions of the present invention may be formulated in a conventional manner using one or more pharmaceutically acceptable carriers. Thus, the active compounds or the active combinations of the invention may be formulated for oral, buccal, intranasal, parenteral (e.g., intravenous, intramuscular, intraperitoneal, or subcutaneous or through an implant) nasal, vaginal, sublingual, rectal o topical administration or in a form suitable for administration by inhalation or insufflation.

For oral administration, the pharmaceutical compositions may take the form of, for example, tablets or capsules prepared by conventional means with pharmaceutically acceptable excipients such as binding agents such as pregelatinized maize starch, polyvinylpyrrolidone or hydroxypropyl methylcellulose; fillers such as lactose, microcrystalline cellulose or calcium phosphate; lubricants such as magnesium stearate, talc or silica; disintegrants such as potato starch or sodium starch glycolate; or wetting agents such as sodium lauryl sulphate. The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents such as sorbitol syrup, methyl cellulose or hydrogenated edible fats; emulsifying agents such as lecithin or acacia, non-aqueous vehicles such as almond oil, oily esters or ethyl alcohol; and preservatives such as methyl or propyl p-hydroxybenzoates or sorbic acid.

For buccal administration, the composition may take the form of tablets or lozenges formulated in conventional manner.

The active compounds or the active combinations of the invention may be formulated for parenteral administration by injection, including using conventional catheterization techniques or infusion. Formulations for injection may be presented in unit dosage form, for example, in ampoules or in multi-dose containers, with an added preservative. The compositions containing the active combinations may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, for example, sterile pyrogen-free water, before use.

The active compounds or the active combinations of the invention may also be formulated in rectal compositions such as suppositories or retention enemas, for example, containing conventional suppository bases such as cocoa butter or other glycerides. Compositions for vaginal administration are preferably suppositories that may contain, in addition to the active substance, excipients such as cocoa butter or a suppository wax. Compositions for nasal or sublingual administration are also prepared with standard excipients well known in the art.

For intranasal administration or administration by inhalation, the active compounds or the active combinations of the invention are conveniently delivered in the form of a solution or suspension from a pump spray container that is squeezed or pumped by the patient or as an aerosol spray presentation from a pressurized container or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compounds or the active combinations. Capsules and cartridges, made, for example, from gelatin, for use in an inhaler or insulator may be formulated containing a powder mix of an active compound and a suitable powder base such as lactose or starch.

The pharmaceutical compositions of the present invention can consist of a combination of immediate release and controlled release characteristics. Such compositions can take the form of combinations of the active ingredients that range in size from nanoparticles to microparticles or in the form of a plurality of pellets with different release rates. The tablet or capsule composition of the present invention can contain a 5-HT_{1B} receptor antagonist of the formula I or II in sustained or controlled release form and the γ-aminobutyric acid modulator in an immediate release form. Alternatively, the 5-HT_{1B} receptor antagonist can be in immediate release form and the γ-aminobutyric acid modulator can be in sustained or controlled release form.

An exemplary dose of the active combinations of the invention for oral, parenteral or buccal administration to the average adult human for the treatment of the conditions referred to above, such as depression, is about 0.1 to about 200 mg of the active compound of formula I or II and of about 0.1 to about 100 mg of the γ-aminobutyric acid modulator per unit dose which could be administered, for example, 1 to 4 times per day.

The composition of this invention may contain, for example, gabapentin; pregabalin; or a pharmaceutically acceptable salt thereof as the γ-aminobutyric acid modulator and 4-benzyl-2-[2-(4-methylpiperazin-1-yl)-benzylidene]-thiomorpholin-3-one, 4-(3,4-dichlorophenyl)-2-[2-(4-methylpiperazin-1-yl)-benzylidene]-thiomorpholin-3-one, or 2-[2-(4-methylpiperazin-1-yl)-benzylidene]-4-(4-trifluoromethyl-phenyl)-thiomorpholin-3-one as the 5-HT_{1B} antagonist. An exemplary daily dose of the γ-aminobutyric acid modulator in a pharmaceutical composition of this invention for oral, parenteral, rectal or buccal administration to the average adult human for the treatment of the conditions referred to above is from about 0.1 to about 300 mg of γ-aminobutyric acid modulator per unit dose administered 1 to 3 times per day. Exemplary and preferred doses for γ-aminobutyric acid modulators are determined on a compound by compound basis. 4-benzyl-2-[2-(4-methylpiperazin-1-yl)-benzylidene]-thiomorpholin-3-one, 4-(3,4-dichlorophenyl)-2-[2-(4-methylpiperazin-1-yl)-benzylidene]-thiomorpholin-3-one, or 2-[2-(4-methylpiperazin-1-yl)-benzylidene]-4-(4-trifluoromethyl-phenyl)-thiomorpholin-3-one may each be present in an amount between about 0.1 and about 200 mg, preferably about 0.3 to about 100 mg.

Aerosol formulations for treatment of the conditions referred to above, for example, migraine, in the average adult human are preferably arranged so that each metered dose or “puff” of aerosol contains about 20 μg to about 1000 μg of the compound of formula I or II. The overall daily dose with an aerosol will be within the range about 100 μg to about 10 mg. Administration may be several times daily, for example 2, 3, 4 or 8 times, giving for example, 1, 2 or 3 doses each time. Aerosol formulations containing a compound of formula I or II and a γ-aminobutyric acid modulator for treatment of the conditions referred to above in the average adult human are preferably arranged so that each metered dose or “puff” of aerosol contains about 100 μg to about 10,000 μg of the compound of formula I formula I or Ii and about 100 μg to about 30,000 μg of the γ-aminobutyric acid modulator. The overall daily dose with an aerosol will be within the range about 100 μg to about 20,000 μg of the compound of formula I or II and about 100 μg to about 60,000 μg of the γ-aminobutyric acid modulator. Administration may be several times daily, for example 1, 3, 4 or 8 times, giving for example, 1, 2 or 3 doses each time.

The γ-aminobutyric acid modulator and the 5-HT_{1B} receptor antagonists of formula I or II may be administered either alone or in combination with pharmaceutically acceptable carriers by either of the routes previously indicated, and such administration can be carried out in both single and multiple dosages. More particularly, this active combination can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I or II are present in such dosage forms at concentration levels ranging from about 0.1% to about 95% by weight of the total composition, i.e., in amounts which are sufficient to provide the desired unit dosage, and a γ-aminobutyric acid modulator is present in such dosage forms at concentration levels ranging from about 0.1% to about 95% by weight of the total composition, i.e., in amounts which are sufficient to provide the desired unit dosage.

The γ-aminobutyric acid modulator and the 5-HT_{1B} receptor antagonists of formula I or II may be administered together or separately. When administered separately, the γ-aminobutyric acid modulators and the compounds of formula I or II may be administered in either order, provided that after administration of the first of the two active ingredients, the second active ingredient is administered within 24 hours or less, preferably 12 hours or less.

A preferred dose ratio of a γ-aminobutyric acid modulator to a compound of formula I or II in the active combination formulation for oral, parenteral or buccal administration to the average adult human for the treatment of the conditions referred to above is from about 0.001 to about 1000, preferably from about 0.01 to about 100.

When referring to these preformulation compositions as homogeneous, it is meant that the active ingredients is dispersed evenly throughout the composition so that the composition may be readily subdivided into equally effective unit dosage forms such as tablets, pills and capsules. This solid preformulation composition is then subdivided into unit dosage forms of the type described above containing from about 0.1 to about 2000 mg of each of the active ingredients of the present invention. Typical unit dosage forms contain from about 1 to about 300 mg, for example about 1, 2, 5, 10, 25, 50 or 100 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The dosage of active ingredients in the compositions and methods of this invention may be varied; however, it is necessary that the amount of the active ingredients in such compositions be such that a suitable dosage form is obtained. The selected dosage depends upon the desired therapeutic effect, on the route of administration, the particular compounds administered, the duration of the treatment, and other factors. All dosage ranges and dosage levels mentioned herein refer to each pharmaceutically active compound present in the pharmaceutical compositions and kits of the present invention, as well as those used in the methods of the present invention. Generally, dosage levels of between about 0.01 and about 100 mg/kg of body weight daily are administered to humans and other animals, e.g., mammals. A preferred dosage range in humans is about 0.1 to about 50 mg/kg of body weight daily which can be administered as a single dose or divided into multiple doses. A preferred dosage range in mammals other than humans is about 0.01 to about 10.0 mg/kg of body weight daily which can be administered as a single dose or divided into multiple doses. A more preferred dosage range in mammals other than humans is about 0.1 to about 5.0 mg/kg of body weight daily which can be administered as a single dose or divided into multiple doses.

In general, the pharmaceutical compositions, methods and kits of this invention, will be administered at dosages of a therapeutically effective amount of the first and of the second active compound in single or divided doses. The term “therapeutically effective amount” as used herein refers to a sufficient amount of the compound to treat the disorders and disorders or conditions disclosed herein at a reasonable benefit/risk ratio applicable to any medical treatment.

The specific therapeutically effective dose level for any particular patient will depend upon a variety of factors including the disorder being treated and the severity of the disorder; activity of the specific compound employed; the specific composition employed; the age. However, some variation in dosage will necessarily occur depending upon the condition of the subject being treated. The person responsible for administration will, in any event, determine the appropriate dose for the individual subject.

The dosage amounts set forth in this description and in the appendant claims may be used, for example, for an average human subject having a weight of about 65 kg to about 70 kg. The skilled practitioner will readily be able to determine any variation in the dosage amount that may be required for a subject whose weight falls outside the about 65 kg to about 70 kg range, based upon the medical history of the subject. The pharmaceutical combinations may be administered on a regimen of up to 6 times per day, preferably 1 to 3 times per day, such as 2 times per day or once daily.

The present invention also encompasses treatment with a combination of active ingredients which may be administered separately. Accordingly, the invention also relates to combining separate pharmaceutical compositions in kit form. Thus, in one embodiment, the kit comprises two separate pharmaceutical compositions: a γ-aminobutyric acid modulator or a pharmaceutically acceptable salt of said γ-aminobutyric acid modulator; and a 5-HT_{1B} receptor antagonist of the formula I or II or a pharmaceutically acceptable salt of said 5-HT_{1B} receptor antagonist. The kit also comprises a container for containing the separate compositions such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms, such as tablets, capsules, and the like. It may be desirable to provide a memory aid on the kit, e.g., in the form of numbers next to the tablets or capsules whereby the numbers correspond with the days of the regimen which the dosage form so specified should be ingested. Another example of such a memory aid is a calendar printed on the card e.g., as follows “First Week, Monday, Tuesday, . . . etc. . . . Second Week, Monday, Tuesday, . . . etc. Other variations of memory aids will be readily apparent. A “daily dose” can be a single tablet or capsule or several tablets or capsules to be taken on a given day. Also, a daily dose of a γ-aminobutyric acid modulator, or a pharmaceutically acceptable salt of said γ-aminobutyric acid modulator can consist of one tablet or capsule, while a daily dose of the 5-HT_{1B} receptor antagonist of formula I or II or pharmaceutically acceptable salt of said 5-HT_{1B} receptor antagonist can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

In another embodiment, the present invention comprises kits comprising a pharmaceutical composition, a package, and a package insert. The pharmaceutical composition of these kits contains either a γ-aminobutyric acid modulator or a 5-HT_{1B} receptor antagonist of formula I or II. The kits of the present invention containing a pharmaceutical composition containing a γ-aminobutyric acid modulator differ from known kits containing a pharmaceutical composition containing a γ-aminobutyric acid modulator in that on the package and/or on the package insert of the kits it is stated that the pharmaceutical composition is to be administered together with a pharmaceutical composition containing a 5-HT_{1B} receptor antagonist. The kits of the present invention containing a pharmaceutical composition containing a 5-HT_{1B} receptor antagonist of formula I or II differ from known kits containing a pharmaceutical composition containing a 5-HT_{1B} receptor antagonist in that on the package and/or on the package insert of the kits it is stated that the pharmaceutical composition is to be administered together with a pharmaceutical composition containing a γ-aminobutyric acid modulator.

The term “together with” as used in the immediately preceding paragraph is intended to encompass the simultaneous administration of the two pharmaceutical compositions (e.g., a tablet containing one pharmaceutical composition is to be administered orally while the other pharmaceutical composition is administered by way of infusion, two tablets or capsules are to be swallowed together, etc.). The term “together with” is also intended to include the administration of the two pharmaceutical compositions in a specifically timed manner, i.e., one pharmaceutical composition is to be administered a certain time period after administration of the other pharmaceutical composition. The time period in which the two pharmaceutical compositions are to be administered must be sufficiently short for the γ-aminobutyric acid modulator and the 5-HT_{1B} receptor antagonist of formula I or II to exhibit their activity contemporaneously, preferably in a synergistic manner. The exact time period depends on the specific compounds of the pharmaceutical compositions, the application route, the kind and severeness of the disease to be treated, the kind, age, and condition of the patient to be treated, etc., and can be determined by a physician using known methods in combination with the disclosure of the present invention. Generally, the two compositions are to be administered within 24 hours or less, such as 12 hours or less, preferably within 5 hours, more preferably within 2 hours, and even more preferably within one hour. Most preferably, the two compositions are to be administered at the same time or one immediately after the other.

The combinations of this invention, i.e., a γ-aminobutyric acid modulator and a 5-HT_{1B} receptor antagonist, may be tested for conditions such as, for example, migraine, depression, obsessive compulsive disorder, post-traumatic stress disorder (PTSD), and eating disorders according to the procedures.

The invention is further illustrated by, but by no means limited to, the following examples.

### EXAMPLE 1

A pharmaceutical composition is prepared by combining 4-benzyl-2-[2-(4-methylpiperazin-1-yl)-benzylidene]-thiomorpholin-3-one, 4-(3,4-dichlorophenyl)-2-[2-(4-methylpiperazin-1-yl)-benzylidene]-thiomorpholin-3-one, or 2-[2-(4-methylpiperazin-1-yl)-benzylidene]-4-(4-trifluoromethyl-phenyl)-thiomorpholin-3-one as the 5-HT_{1B} receptor antagonist with a γ-aminobutyric acid modulator that is either gabapentin or pregabalin in a pharmaceutically acceptable carrier. The composition contains about 1.0 mg to about 160 mg of the 5-HT_{1B} receptor antagonist and about 5 mg to about 200 mg of the γ-aminobutyric acid modulator to deliver on a daily basis The composition is administered to a patient for the treatment of depression on a daily, twice daily, or three times daily basis.

It should be understood that the present invention is not limited to the embodiments described herein. Numerous modifications can be made by one skilled in the art having the benefits of the teachings given here. Such modifications should be taken as being encompassed within the scope of the present invention as set forth in the appended claims.

1. A pharmaceutical composition comprising
(i) a γ-aminobutyric acid modulator or a pharmaceutically acceptable salt thereof,
(ii) a 5-HT_{1B} receptor antagonist or a pharmaceutically acceptable salt thereof;
wherein the 5-HT_{1B} receptor antagonist is selected from the group consisting of
(A) a compound of the formula I 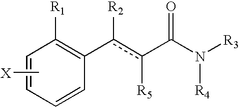
wherein, in formula l:
R¹ is a group of the formula G¹, G², G³, G⁴, G⁵, G⁶ or G⁷ depicted below, 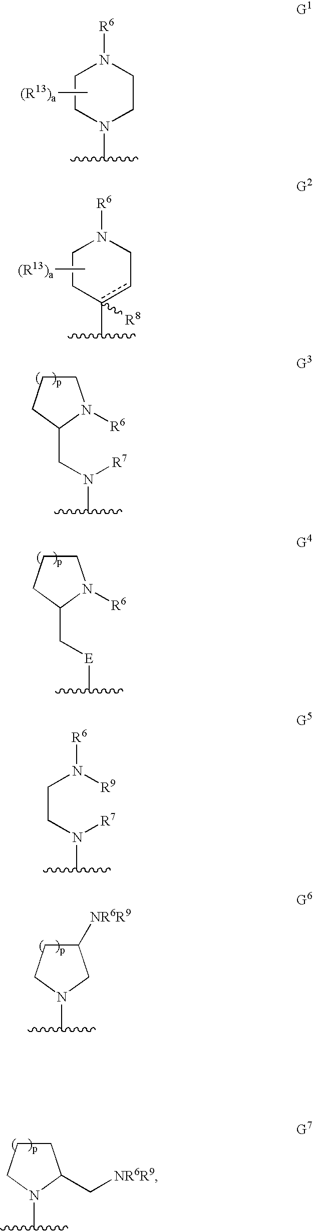
a is zero to eight;
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
E is oxygen, sulfur, SO or SO₂;
X is hydrogen, chloro, fluoro, bromo, iodo, cyano, (C₁-C₆)alkyl, hydroxy trifluoromethyl, (C₁-C₆)alkoxy, —SOₜ(C₁-C₆)alkyl wherein t is zero one or two, —CO₂R¹⁰ or —CONR¹¹R¹²,
R² is hydrogen, (C₁-C₄)alkyl, phenyl or naphthyl, wherein said phenyl or naphthyl is optionally substituted with one or more substituents independently selected from the group consisting of chloro, fluoro, bromo, iodo, (C₁-C₆)alkyl, (C₁-C₆)alkoxy, trifluoromethyl, cyano and —SOₖ(C₁-C₆)alkyl wherein k is zero, one or two;
R³ is —(CH₂)ₘB, wherein m is zero, one, two or three and B is hydrogen, phenyl, naphthyl or a 5 or 6 membered heteroaryl group containing from one to four hetero-atoms in the ring, and wherein each of the foregoing phenyl, naphthyl and heteroaryl groups is optionally substituted with one or more substituents independently selected from the group consisting of chloro, fluoro, bromo, iodo, (C₁-C₆)alkyl, (C₁-C₆)alkoxy, (C₁-C₆) alkoxy-(C₁-C₆)alkyl-, trifluoromethyl, trifluoromethoxy, cyano, hydroxy, —COOH and —SOₙ(C₁-C₆)alkyl wherein n is zero, one or two;
R⁴ is (C₁-C₆)alkyl or C₆-C₁₀ aryl;
or R3 and R⁴ may optionally be taken together with the nitrogen to which they are attached to form a five to seven membered heteroalkyl ring, wherein any two of the carbon atoms of said heteroalkyl ring is optionally replaced with a heteroatom selected from the group consisting of nitrogen, oxygen or sulfur;
R⁵ is hydrogen, (C₁-C₆)alkyl or aryl, wherein aryl is selected from the group consisting of phenyl or naphthyl, wherein any of said aryl is optionally independently substituted on any available bonding site by any of the radicals of X;
or R⁵ and R⁴ taken together form a divalent group —Yₙ₂—;
Y is selected from the group consisting of (a) CR⁴R⁵, wherein R⁴ and R⁵ are independently selected from hydrogen, (C₁-C₆)alkyl and trifluoromethyl; (b) a phenylene, naphthylene or a 5 or 6 membered heteroarylene ring comprising from one to four hetero-atoms in the heteroarylene ring, and wherein each of the foregoing phenylene, naphthylene and heteroarylene rings may optionally be substituted with one or more substituents independently selected from the group consisting of chloro, fluoro, bromo, iodo, (C₁-C₆)alkyl, (C₁-C₆)alkoxy, (C₁-C₆) alkoxy-(C₁-C₆)alkyl-, trifluoromethyl, trifluoromethoxy, cyano, hydroxy, —COOH and —SOₙ(C₁-C₆)alkyl wherein n is zero, one or two, wherein two adjacent ring atoms of ring Y are also ring atoms of ring A; and (c) an optionally substituted (C₁-C₄) heteroalkyl bridge that, together with the atoms to which it is attached, forms a five to seven membered heterocycle containing two to four heteroatoms selected from the group consisting of 1,3-oxazolidin-4-on-5-yl, 1,3-oxazolidin-2,4-dion-5-yl, 4,5-dihydro-1,2-oxazolidin-3-on-4-yl, 1,3-thiazolidin-4-on-5-yl, 1,3-thiazolidin-2,4-dion-5-yl, 1,3-pyrazolidin-4-on-5-yl, 1,3-imidazolidin-2,4-dion-5-yl, 1,2-pyrazolidin-3-on-4-yl, 1,2-thiazolidin-1,1,3-trion-4-yl, 1,2-thiazolidin-3-on-4-yl, tetrahydro-1,2-oxazin-3-on-4-yl, tetrahydro-1,3-oxazin-4-on-5-yl, tetrahydro-1,3-oxazin-2,4-dion-5-yl, morpholin-3-on-2-yl, morpholin-3,5-dion-2-yl, 2,3-dihydro-1,4-oxazin-3-on-2-yl, tetrahydro-1,3-thiazin-4-on-5-yl, tetrahydro-1,3-thiazin-2,4-dion-5-yl, tetrahydro-1,2-thiazin-3-on-4-yl, thiomorpholin-3-on-2-yl, thiomorpholin-3,5-dion-2-yl, 2,3-dihydro-1,4-thiazin-3-on-2-yl, hexahydro-1,2-diazin-3-on-4-yl, 4, 5-dihydro-2H-pyridazin-3-on-4-yl, hexahydro-1,3-diazin-4-on-5-yl, hexahydro-1,3-diazin-2,4-dion-5-yl, piperazin-2-on-3-yl, piperazin-2,6-dion-3-yl, tetrahydro-1,3,4-thiadiazin-5-on-6-yl, 5,6-dihydro-1,3,4-thiadiazin-5-on-6-yl, 1,3,4-oxadiazin-5-on-6-yl, 5,6-dihydro-1,2,4-oxadiazin-5-on-6-yl, tetrahydro-1,2,4-oxadiazin-5-on-6-yl, 1,2,4-triazin-5-on-6-yl, tetrahydro-1,2,4-oxadiazin-5-on-6-yl, 5,6-dihydro-1,2,4-oxadiazin-5-on-6-yl, 1,2,4-oxadiazin-3,5-dion-6-yl, 1,2,4-trazin-6-on-5-yl, hexahydro-1,2-oxazepin-3-on-2-yl, hexahydro-1,3-oxazepin-4-on-5-yl, hexahydro-1,4-oxazepin-3-on-2-yl, hexahydro-1,4-oxazepin-3,5-dion-2-yl, hexahydro-1,4-oxazepin-3,5-dion-6-yl, 2,3,5,6-tetrahydro-1,4-oxazepin-5,7-dion-6-yl, hexahydro-1,4-oxazepin-5-on-6-yl, hexahydro-1,3-oxazepin-2,4-dion-5-yl, hexahydro-1,2-thiazepin-3-on-4-yl, hexahydro-1,4-thiazepin-3-on-2-yl, 2,3,4,5-tetrahydro-1,4-thiazepin-3-on-2-yl, hexahydro-1,4-thiazepin-3, 5-dion-2-yl, hexahydro-1,4-thiazepin-3,5-dion-6-yl, 2,3,6,7-tetrahydro-1,4-thiazepin-5-on-6-yl, 6,7-dihydro-1,4-thiazepin-5-on-6-yl, hexahydro-1,3-thiazepin-2,4-dion-5-yl, hexahydro-1,2-diazepin-3-on-4-yl, hexahydro-1,3-diazepin-2,4-dion-5-yl, hexahydro-1,4-diazepin-2-on-3-yl, hexahydro-1,4-diazepin-5-on-6-yl, hexahydro-1,4-diazepin-5,7-dion-6-yl, hexahydro-1,3, 5-thiadiazepin-3-on-7-yl, 4,5,6,7-tetrahydro-1-3,5-thiadiazepin-6-on-7-yl, and 2,3,5,6-tetrahydro-1,2,4-triazepin-3,5-dion-7-yl; wherein the substituents on any of the carbon atoms capable of supporting an additional bond, of said (C₁-C₄) heteroalkyl bridge, are chloro, fluoro, (C₁-C₆)alkyl, (C₁-C₆)alkoxy, trifluoromethyl or cyano; wherein the substituents on any of the nitrogen atoms capable of supporting an additional bond, of said (C₁-C₄) heteroalkyl bridge, are (C₁-C₆)alkyl or trifluoromethyl,
n2 is one, two, three or four, with the proviso that n2 is one when Y is not CR⁴R⁵;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a C₂-C₄ alkylene chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
and p is one, two, or three;
each of R¹⁰, R¹¹ and R¹² is selected, independently, from the groups set forth in the definition of R²; or R¹¹ and R¹², together with the nitrogen to which they are attached, form a 5 to 7 membered heteroalkyl ring that may contain, in addition to the nitrogen atom to which R¹¹ and R¹² are attached, from zero to four heteroatoms selected from the group consisting of nitrogen, sulfur and oxygen, and
the broken lines indicate optional double bonds, with the proviso that when the broken line in G² is a double bond, R⁸ is absent;
(B) a compound of the formula II 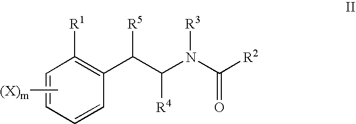
wherein in Formula II,
R¹ is a group of the formula G¹, G², G³, G⁴, G⁸ or G^{6,} wherein G¹, G², G³, G⁴, and G⁶ are each defined as for formula I, and G⁸ is depicted below 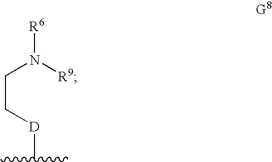
m is 0, 1, 2, 3 or 4;
D is oxygen, sulfur, SO, SO₂, or NR⁷;
a is zero to eight;
p is 1, 2 or 3;
E is oxygen, sulfur, SO or SO₂;
X is hydrogen, chloro, fluoro, bromo, iodo, cyano, (C₁-C₆)alkyl, hydroxy, trifluoromethyl, (C₁-C₆)alkoxy, —S(O)ₜ(C₁-C₆)alkyl wherein t is 0, 1 or 2, —CO₂R¹⁰ or —CON R¹¹R¹²;
R² is —(CH₂)ₜB, wherein t is 0, 1, 2 or 3, and B is hydrogen, phenyl, naphthyl or a 5 or 6 membered heteroaryl group containing from one to four heteroatoms in the ring, and wherein each of the foregoing phenyl, naphthyl and heteroaryl groups may optionally be substituted with one or more substituents independently selected from chloro, fluoro, bromo, iodo, (C₁-C₆)alkyl, (C₁-C₆)alkoxy, (C₁-C₆)alkoxy-(C₁-C₆)alkyl-, trifluoromethyl, trifluoromethoxy, cyano, hydroxy, —COOH and —SOₙ(C₁-C₆)alkyl wherein n is 0, 1 or 2;
R³ and R⁴ are each independently hydrogen, (C₁-C₄)alkyl or —(CH₂)_{q}-J wherein q is 0, 1, 2 or 3, and J is phenyl or naphthyl, wherein said phenyl or naphthyl may be optionally substituted with one to three substituents independently selected from the group consisting of chloro, fluoro, bromo, iodo, (C₁-C₆)alkyl, (C₁-C₆)alkoxy, trifluoromethyl, cyano and —S(O)ₖ(C₁-C₆)alkyl wherein k is 0, 1 or 2;
R⁵ is hydrogen or (C₁-C₃)alkyl;
R⁶ is selected from the group consisting of hydrogen, (C₁-C₆)alkyl optionally substituted with (C₁-C₆)alkoxy or one to three fluorine atoms, or [(C₁-C₄)alkyl]aryl wherein the aryl moiety is phenyl, naphthyl, or heteroaryl-(CH₂)_{q2}—, wherein the heteroaryl moiety is selected from the group consisting of pyridyl, pyrimidyl, benzoxazolyl, benzothiazolyl, benzisoxazolyl and benzisothiazolyl and q2 is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, cyano and —SO_{g}(C₁-C₆)alkyl, wherein g is zero, one or two;
R⁷ is selected from the group consisting of hydrogen, (C₁-C₆)alkyl, [(C₁-C₄)alkyl]aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl, benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, (C₁-C₆)alkyl, (C₁-C₆)alkoxy, trifluoromethyl, —C(═O)—(C₁-C₆)alkyl, cyano and —SOⱼ(C₁-C₆)alkyl, wherein j is zero, one or two;
or R⁶ and R⁷ taken together form a 2 to 4 carbon chain;
R⁸ is hydrogen or (C₁-C₃)alkyl;
R⁹ is hydrogen or (C₁-C₆)alkyl;
or R⁶ and R⁹, together with the nitrogen atom to which they are attached, form a 5 to 7 membered heteroalkyl ring that contains, in addition to the nitrogen atom to which R⁶ and R⁹ are attached, from zero to four heteroatoms selected from the group consisting of nitrogen, sulfur and oxygen;
each of R¹⁰, R¹¹ and R¹² is selected, independently, from the groups set forth in the definition of R³; or R¹¹ and R¹², together with the nitrogen to which they are attached, form a 5 to 7 membered heteroalkyl ring that may contain, in addition to the nitrogen atom to which R¹¹ and R¹² are attached, from zero to four heteroatoms selected from the group consisting of nitrogen, sulfur and oxygen, and
each R¹³ is, independently, (C₁-C₄)alkyl or a (C₁-C₄)methylene bridge from one of the ring carbons of the piperazine or piperidine ring of G¹ or G², respectively, to the same or another ring carbon or a ring nitrogen of the piperazine or piperidine ring of G¹ or G² respectively, having an available bonding site, or to a ring carbon of R⁶ having an available bonding site;
with the proviso that when B is hydrogen, t is not zero; and
with the proviso that when the broken line in formula G² is a double bond, R⁸ is absent;
and optionally
(iii) a pharmaceutically acceptable carrier.

2. The composition of claim 1, wherein in formula I R¹ is 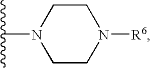
R⁶ is (C₁-C₆)alkyl and R²is hydrogen.

3. The composition of claim 1, wherein in formula I R³ is hydrogen, phenyl or benzyl optionally substituted by chloro, fluoro, bromo, iodo, (C₁-C₆)alkyl or trifluoromethyl.

4. The composition of claim 1, wherein in formula I R⁴ is hydrogen or (C₁-C₆)alkyl.

5. The composition of claim 1, wherein in formula I: R¹ is 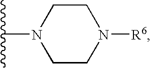
R⁶ is (C₁-C₆)alkyl and R2 is hydrogen; R³ is phenyl or benzyl optionally substituted by chloro, fluoro, bromo, iodo, (C₁-C₆)alkyl or trifluoromethyl; and R⁴ is hydrogen or (C₁-C₆)alkyl.

6. The composition of claim 1, wherein in formula I R⁴ and R⁵, together with the nitrogen to which they are attached, form a 5 to 7 membered heteroalkyl ring that is selected from the group consisting of pyrrolidine, isoxazolidine, 1,3-oxazolidin-3-yl, isothiazolidine, 1,3-thiazolidin-3-yl, 1,2-pyrazolidin-2-yl, 1,3-pyrazolidin-1-yl, piperidine, thiomorpholine, 1,2-tetrahydrothiazin-2-yl, 1,3-tetrahydrothiazin-3-yl, tetrahydrothiadiazine, morpholine, 1,2-tetrahydrodiazin-2-yl, 1,3-tetrahydrodiazin-1-yl, and piperazine.

7. The composition of claim 1, wherein in formula I m is 0 or 1.

8. The composition of claim 1, wherein in formula II, R¹ is 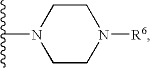
R⁶ is (C₁-C₆)alkyl and R³is hydrogen.

9. The composition of claim 1, wherein in formula II, R¹ is 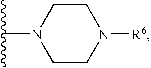
R⁶ is (C₁-C₆)alkyl and R³ is hydrogen.

10. The composition of claim 1, wherein in formula II, R¹ is 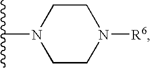
R⁶ is (C₁-C₆)alkyl and R³ is hydrogen; R²is phenyl or benzyl optionally substituted by chloro, fluoro, bromo, iodo, (C₁-C₆)alkyl or trifluoromethyl; and R⁴ is hydrogen or (C₁-C₆)alkyl.

11. The composition of claim 1, wherein the 5-HT_{1B} antagonist is selected from the group consisting of
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thiomorpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzylidene]-thiomorpholin-3-one; and
4-(3,4-dichlorophenyl)-2-[2-(4-methylpiperazin-1-yl)-benzylidene]-thiomorpholin-3-one;
or a pharmaceutically acceptable salt thereof.

12. The composition of claim 1, wherein the γ-aminobutyric acid modulator is a compound of the formula 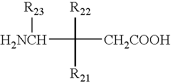
wherein R₂₁ is a straight or branched alkyl group having from 1 to 6 carbon atoms, phenyl, or cycloalkyl having from 3 to 6 carbon atoms; R₂₂ is hydrogen or methyl; and R₂₃ is hydrogen, methyl or carboxyl; and the pharmaceutically acceptable salts thereof.

13. The composition of claim 1, wherein the γ-aminobutyric acid modulator is a compound of the formula 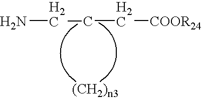
wherein R₂₄ is a hydrogen atom or a C₁-C₆ alkyl radical and n3 is 4,5, or 6; and the pharmaceutically acceptable salts thereof.

14. The composition of claim 1, wherein the γ-aminobutyric acid modulator is selected from the group consisting of muscimol, progabide, riluzole, baclofen, gabapentin, vigabatrin, tiagabine, lamotrigine, pregabalin, topiramate, and a pharmaceutically acceptable salt thereof.

15. The composition of claim 14, wherein the γ-aminobutyric acid modulator is selected from the group consisting of gabapentin, pregabalin, and a pharmaceutically acceptable salt thereof.

16. A method for treating a disorder or condition selected from the group consisting of hypertension, depression, generalized anxiety disorder, phobias, posttraumatic stress disorder, avoidant personality disorder, sexual dysfunction, eating disorders, obesity, chemical dependencies, cluster headache, migraine, pain, Alzheimer's disease, obsessive-compulsive disorder, panic disorder, memory disorders, Parkinson's diseases, endocrine disorders, cerebellar ataxia, gastrointestinal tract disorders, negative symptoms of schizophrenia, premenstrual syndrome, Fibromyalgia Syndrome, stress incontinence, Tourette syndrome, trichotillomania, kleptomania, male impotence, cancer, chronic paroxysmal hemicrania and headache in a mammal, comprising administering to a mammal in need of such treatment components (i) and (ii) as defined in claim 1.

17. The method of claim 16, wherein component (i) is selected from the group consisting of muscimol, progabide, riluzole, baclofen, gabapentin, vigabatrin, tiagabine, lamotrigine, pregabalin, topiramate, and a pharmaceutically acceptable salt thereof.

18. The method of claim 16, wherein component (ii) is selected from the group consisting of
4-benzyl-2-[2-(4-methylpiperazin-1-yl)-benzylidene]-thiomorpholin-3-one;
4-(3,4-dichlorobenzyl)-2-[2-(4-methylpiperazin-1-yl)-benzylidene]-thiomorpholin-3-one;
2-[2-(4-methylpiperazin-1-yl)-benzylidene]-4-(4-trifluoromethylphenyl)-thiomorpholin-3-one;
2-[2-(4-methylpiperazin-1-yl)-benzylidene]-thiomorpholin-3-one;
4-(3,4-dichlorophenyl)-2-[2-fluoro-6-(4-methylpiperazin-1-yl)-benzylidene]-thiomorpholin-3-one; and
4-(3,4-dichlorophenyl)-2-[2-(4-methylpiperazin-1-yl)-benzylidene]-thiomorpholin-3-one;
and a pharmaceutically acceptable salt thereof.

19. A method for treating a disorder or condition that can be treated by enhancing serotonergic neurotransmission in a mammal, comprising administering to a mammal in need of such treatment components (i) and (ii) as defined in claim 1.

20. The method of claim 19 further comprising administering a 5-HT_{1A} antagonist or a pharmaceutically acceptable salt thereof, wherein the amounts of each of components (i), (ii) and the 5-HT_{1A} antagonist or a pharmaceutically acceptable salt thereof are such that the combination of components (i), (ii) and the 5-HT_{1A} antagonist or a pharmaceutically acceptable salt thereof is effective in treating the disorder or condition.
